(19) United States
(12) Patent Application Publication
(10) Pub. No.: US 2023/0123183 A1
(43) Pub. Date: Apr. 20, 2023

(54) LYMPHATIC FLUID FOR DIAGNOSTICS

(71) Applicant: DROPLET BIOSCIENCES, INC., Melrose, MA (US)

(72) Inventors: Jose P. Zevallos, St. Louis, MO (US); Aadel Chaudhuri, Chesterfield, MO (US); Stanley N. Lapidus, Golden, CO (US)

(21) Appl. No.: 18/087,185

(22) Filed: Dec. 22, 2022

Related U.S. Application Data
(63) Continuation-in-part of application No. 17/947,871, filed on Sep. 19, 2022.
(60) Provisional application No. 63/246,254, filed on Sep. 20, 2021.

(51) Int. Cl. G01N 33/574; A61P 35/00

(52) U.S. Cl. CPC G01N 33/57492; A61P 35/00; G01N 2800/52

(57) ABSTRACT

The invention related to diagnostic methods for identifying indicia of cancer in fluid collected from a lymphatic channel.

### FIELD OF INVENTION

The invention related to diagnostic methods for identifying indicia of cancer in fluid collected from a lymphatic channel.

### BACKGROUND

Cancer is a leading cause of death globally. Early detection, while beneficial for most cancers, is often difficult. In part, this is because many cancers first develop without presenting any specific clinical symptoms, and diagnosis only occurs when the disease has reached a stage when it is difficult to treat.

Cancer detection has focused on liquid biopsy in blood or plasma for the detection of cell-free tumor DNA. Blood is of high clinical interest because of its accessibility. Unfortunately, many of these methods lack sensitivity. As a result, early cancer detection, when tumor DNA is present as only a minute fraction of the DNA collected from blood or plasma, is often difficult. Moreover, due to the lack of sensitivity, progression of the disease and its response to therapeutic intervention are difficult to monitor.

Tissue, such as tumor tissue, generally is the most informative sample for diagnosis and prognosis of cancer. Unfortunately, tissue samples are often difficult to access and subject to limited availability, especially without performing an invasive procedure. In the context of cancer, often by the time tumors are detected, cancer has spread or progressed.

Consequently, physicians and patients are often unable to make timely, informed decisions regarding therapeutic intervention.

### SUMMARY

The present invention provides methods for collecting and testing fluid from lymphatic channels for indicia of cancer. Preferred methods comprise the steps of collecting fluid from a lymphatic channel of a patient and identifying indicia of cancer in the fluid. The present invention is useful for the detection of cancer in a patient prior to presentation of symptoms, allowing for the early detection of a cancer.

The present invention is based on the discovery that fluid collected from a lymphatic channels presents a rich source of diagnostic content. Lymphatic channel fluid allows for detection of cancer biomarkers, even when their concentration is low.

Accordingly, the present invention provides methods for disease diagnosis comprising the steps of collecting lymphatic channel fluid and identifying indicia of cancer in the fluid. Lymphatic channel fluid may be analyzed directly or may be extracted from fluid obtained via general drain fluid.

Collection of fluid from lymphatic channels may further provide information regarding the location of a tumor. For example, the presence of tumor biomarkers in lymphatic channels may reveal the location of a primary tumor as a result of proximity to the tumor. In addition, the presence of tumor biomarkers in lymphatic channels is an indication of metastasis and may result in further diagnostic investigation (e.g., lymph node extraction). Accordingly, the present invention may comprise collecting fluid from a lymphatic channel located between a tumor and a first lymph node. The invention may further comprise the step of identifying indicia of in-transit metastases.

According to the invention, fluid may be collected along any lymphatic channel. For example, when a tumor is suspected of being in the mouth or neck, fluid may be collected at or near lymphatic channels in the neck of the patient. Where a tumor may be suspected of being in the abdomen, for example an abdominal organ, fluid may be collected from the axillary lymph nodes, thoracic duct, or the right lymphatic duct. In addition, collection of fluid from the thoracic duct or right lymphatic duct may allow for the early and general detection of cancer or metastasis throughout the body.

Fluid may be collected from the lymphatic channel by any known method. For example, the step of collecting fluid from the lymphatic channel may comprise cannulating a lymphatic channel of a patient and draining the lymphatic fluid into a collection vessel. The fluid from the lymphatic channel may be collected during a procedure that is unrelated to cancer treatment or detection, and as such, the lymphatic fluid may be collected through a drain, for example a surgical drain, and thereafter collected. The step of collecting fluid from the lymphatic channel may comprise cannulating the lymphatic channel. In aspects of the invention. the collecting step may also simply comprise receiving the sample, for example in a laboratory setting, the sample having been previously collected from a clinical setting.

The present invention may also comprise obtaining and/or analyzing a lymph node for indicia of cancer. This is advantageous because it allows for the further assessment of the relative amounts of cancer biomarkers in the lymphatic channel as compared to a lymph node.

The identification of cancer-related biomarkers is accomplished by any known detection method at the convenience of the skilled artisan. For example, nucleic acids or proteins are detected by sequencing, for example proteomic or nucleic acid sequencing. Accordingly, methods of the present invention may comprise the further step of obtaining a genomic profile from material in the fluid. In addition, tumor-associated genetic material, or any other indicia of cancer, may be identified in the fluid collected from the lymphatic channel without significant isolating or separating steps, for example step of isolation lymphatic fluid.

Biomarkers identified in lymphatic channels may be correlated with the onset, progression, staging and recurrence monitor of cancer; as well as for therapeutic selection and efficacy monitoring.

In some embodiments, biomarkers indicative of cancer may be a ratio of circulating tumor cells to cell-free DNA. An amount of one or more biomarkers identified in lymphatic channel fluid may be compared to an amount in blood, plasma or lymph node tissue from the same subject. An aspect of the invention is that lymphatic channel fluid contains a greater ratio of circulating tumor cells to cell-free DNA than the same volume of blood or plasma.

Collection of fluid from a lymphatic channel may further allow for comparison of indicia of cancer in the lymphatic fluid to reference levels for a healthy subject or for references levels for subjects with varying stages of disease. This allows for the potential to use a single sample collected for diagnosis or staging. Reference data may also be used for normalization and may include phenotypic data, genomic data, proteomic data and the like. Accordingly, methods of the invention include normalizing the indicia of cancer with respect to expected amounts in fluid of a patient without cancer.

Collection of fluid from a lymphatic channel over time further allows for monitoring of disease. For example, fluid may be collected before, during and after treatment in order to assess staging or progression of disease or the efficacy of treatment.

The invention contemplates methods for staging cancer. This may include providing a likelihood of metastasis. For example, likelihood of metastasis may be identified by identifying indicia of cancer in the fluid and determining whether the same indicia of cancer are present in a lymph node or in a blood sample. If the indicia are found in the fluid but not in the blood sample, then it can be determined that the tumor has moved to the lymphatic channel of the subject but not yet the blood of the subject. Identifying and tracking the movement of the indicia of cancer in the subject can then be used as a predictor of metastatic disease. The invention also contemplates the identification of residual disease based on the identification indicia of cancer in lymphatic channel fluid.

The invention also provides methods for assessing or screening patients for efficacy of immunotherapies using drain fluid. Ideally, the drain fluid is produced in proximity to a tumor and is evaluated for biomarkers indicative of response to immunotherapy. Exemplary biomarkers include PD-L1, MMR, and CTLA-4, but any biomarker indicative of efficacy in immunotherapy will work according to the invention, as the insight is that those biomarkers are in drain fluid produced in proximity to a tumor and are reflective of the state of the tumor and its susceptibility to immunotherapy. The immunotherapy can be any immunotherapy, including neoadjuvant therapy, checkpoint inhibitor therapy and others. Immunotherapies can be inhibitors of PD-L1, CLTA-4, or both.

### DETAILED DESCRIPTION

The present invention provides methods for disease diagnosis in lymphatic channel fluid. Preferred methods comprise the steps of collecting fluid from a lymphatic channel of a patient and identifying indicia of cancer in said fluid. The present invention allows for the detection of cancer in a patient prior to presentation of symptoms of cancer, allowing for the early detection of a cancer.

Biomarker or indicia of cancer identified by the present invention may be any known biomarker or indicia of cancer present in lymphatic fluid.

For example, the indicia of cancer may comprise tumor cells, immune cells, bacterial cells, viral host cells, donor organ cells, microvascular cells, cell-free DNA, cell-free RNA, circulating tumor DNA, messenger RNA, exosomes, proteins, hormones, and analytes. The indicia of cancer identified may depend on, for example, a specific patient, pathology, surgery type, and surgery site. By analyzing indicia of cancer in the obtained fluid, methods of the invention may provide diagnostic or prognostic information. For example, by identifying circulating tumor cells or cell-free tumor DNA, cancer may be diagnosed in the subject.

In various aspects, indicia of cancer may be identified and quantified using methods known in the art. Suitable assays include, for example, nucleic acid sequencing, PCR, quantitative PCR, digital droplet PCR, Western blot target capture, proteomics, nucleic acid expression analysis, and antibody screening. For example, assays may include whole genome sequencing, next generation DNA sequencing, next generation RNA sequencing, multiplex PCR, methylation analysis, droplet PCR, droplet cell separation, or any combination thereof.

Advantageously, fluorescent labels may be used to identify biomarkers and indicia of cancer. A fluorescent label or fluorescent probe, is a molecule that is attached chemically to aid in the detection of a biomarker. Fluorescent labeling generally uses a reactive derivative of a fluorescent molecule known as a fluorophore. The fluorophore selectively binds to a specific region or functional group on the biomarker and can be attached chemically or biologically. Any known technique for fluorescent labeling may be used, for example enzymatic labeling, protein labeling, or genetic labeling. Any known fluorophore may also be used. Both the fluorophore and labelling technique may be selected and adjusted based on the indicia of cancer to be identified. The most commonly labelled molecules are antibodies, proteins, amino acids and peptides which are then used as specific probes for detection of a particular target.

Fluorescent labelling may be used to identify and quantify indicia of cancer in the surgical fluid sample without separating the components of the surgical fluid. For example, by providing fluorescent labels directly into the surgical fluid, fluorescent microscopy or a colorimetric assay can be used to identify and quantify the presence of the indicia of cancer from a color change alone. For example, fluorescent labels may be applied to the surgical fluid in the surgical suite during a surgical procedure to provide valuable information to the surgeon.

When quantifying a indicia or cancers and biomarker, barcodes may be added to biomarker to aid in amplification, detection, or differentiation of the biomarker. Barcodes may be added to biomarkers by “tagging” the biomarker with the barcode. Tagging may be performed using any known method for barcode addition, for example direct ligation of barcodes to one or more of the ends of a nucleic acid molecule or protein. Nucleic acid molecules may, for example, be end repaired in order to allow for direct or blunt-ended ligation of the barcodes. Barcodes may also be added to nucleic acid molecules through first or second strand synthesis, for example using capture probes or primers. First and second strand synthesis is advantageously used in RNA analysis to generate tagged DNA molecules.

Unique molecular identifiers are a type of barcode that may be provided to biomarkers in a sample to make each biomarker, together with its barcode, unique, or nearly unique. For example, with regard to nucleic acid molecules, this is accomplished by adding, e.g. by ligation or reverse transcription, one or more UMIs to each nucleic acid molecule such that it is unlikely that any two previously identical nucleic acid molecules, together with their UMIs, have the same sequence. By selecting an appropriate number of UMIs, every nucleic acid molecule in the sample, together with its UMI, will be unique or nearly unique. One strategy for doing so is to provide to a sample of nucleic acid molecules a number of UMIs in excess of the number of starting nucleic acid molecules in the sample. By doing so, each starting nucleic molecule will be provided with different UMIs, therefore making each molecule together with its UMIs unique. However, the number of UMIs provided may be as few as the number of identical nucleic acid molecules in the original sample. For example, where no more than six nucleic acid molecules in a sample are likely to be identical, as few as six different UMIs may be provided, regardless of the number of starting nucleic acid molecules.

UMIs are also advantageous in that they can be useful to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, “Counting Molecules in cell-free DNA and single cells RNA”, Karolinska Institutet, Stockholm Sweden, the contests of which are incorporated herein by reference.

For RNA or mRNA sequencing, sequencing may first comprise the step of preparing a cDNA library from barcoded RNA, for example through reverse transcription, and sequencing the cDNA. cDNA sequencing may advantageously allow for the quantification of gene expression within the single cell, and can be useful to identify characteristics of the single cell to, for example, make a diagnosis, prognosis, or determine drug effectiveness.

Reverse transcription may be performed using without limitation dNTPs (mix of the nucleotides dATP, dCTP, dGTP and dTTP), buffer/s, detergent/s, or solvent/s, as required, and suitable enzyme such as polymerase or reverse transcriptase. The polymerase used may be a DNA polymerase, and may be selected from Taq DNA polymerase, Phusion polymerase (as provided by Thermo Fisher Scientific, Waltham, Mass.), or Q5 polymerase. Nucleic acid amplification reagents are commercially available, and may be purchased from, for example, New England Biolabs, Ipswich, Mass., USA. The reverse transcriptase used in the presently disclosed targeted library preparation method may be for example, maxima reverse transcriptase. In some embodiments, the general parameters of the reverse transcription reaction comprise an incubation of about 15 minutes at 25 degrees and a subsequent incubation of about 90 minutes at 52 degrees.

Reverse transcription may be performed by oligos that have a free, 3′ poly-T region. The 3′ portions of the cDNA capture oligos may include gene-specific sequences or oligomers, for example capture primers to reverse transcribe RNA guides comprising a capture sequence. The oligomers may be random or “not-so-random” (NSR) oligomers (NSROs), such as random hexamers or NSR hexamers. The oligos may include one or more handles such as primer binding sequences cognate to PCR primers that are used in the amplifying step or the sequences of NGS sequencing adaptors. The reverse transcription primers may include template switching oligos (TSOs), which may include poly-G sequences that hybridize to and capture poly-C segments added during reverse transcription.

Reverse transcription of non-polyadenylated RNA may comprise use of a capture sequence and a capture primer or probe. Primer sequences may comprise a binding site, for example a primer sequence that would be expected to hybridize to a complementary sequence, if present, on any nucleic acid molecule released from a cell and provide an initiation site for a reaction. The primer sequence may also be a “universal” primer sequence, i.e. a sequence that is complementary to nucleotide sequences that are very common for a particular set of nucleic acid fragments. Primer sequences may be P5 and P7 primers as provided by Illumina, Inc., San Diego, Calif. The primer sequence may also allow a capture probe to bind to a solid support.

Reverse transcription can also be useful for adding a barcode or a UMI, or both to cDNA. This process may comprise hybridizing the reverse transcription primer to the probe followed by a reverse transcription reaction. The complement of a nucleic acid when aligned need not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Nucleic acid molecules may advantageously be amplified prior to sequencing. Amplification may comprise methods for creating copies of nucleic acids by using thermal cycling to expose reactants to repeated cycles of heating and cooling, and to permit different temperature-dependent reactions (e.g. by Polymerase chain reaction (PCR). Any suitable PCR method known in the art may be used in connection with the presently described methods. Non limiting examples of PCR reactions include real-time PCR, nested PCR, multiplex PCR, quantitative PCR, or touchdown PCR.

Sequencing nucleic acid molecules may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and U.S. Pat. No. 6,911,345, each incorporated by reference.

The conventional pipeline for processing sequencing data includes generating FASTQ-format files that contain reads sequenced from a next generation sequencing platform, aligning these reads to an annotated reference genome, and quantifying expression of genes. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

### EXAMPLES

### Example 1

A cannula is inserted into the lymphatic canal downstream of the neck of the subject and upstream of a lymph node of a subject suspected of having oropharyngeal cancer. Fluid samples are collected from the cannula and the fluid samples are centrifuged and filtered. A nuclease, such as EDTA is added to each sample.

Indicia associated with oropharyngeal cancer are isolated and measured from the samples, which include tumor-associated genetic material. The tumor-associated genetic material includes, for example, one or more of cell-free nucleic acids, nucleic acids from a tumor, nucleic acids from an isolated exosome, and/or viral nucleic acids.

Once isolated, the tumor-associated genetic material is analyzed using one or more of nucleic acid sequencing, PCR, and/or Western blot.

This analysis of tumor-associated genetic material provides results that may include, for example, quantities of detected nucleic acids, mutations, variants, copy number, and expression patterns. These results may be compared with other bioassay results, either for other biomarkers in the fluid from the lymphatic duct and/or from a different sample type, such as blood or plasma.

Using these results, one or more scores are produced indicative of the subjects' conditions, disease states, and prognosis. The scores provide a practitioner with valuable insight as to whether to pursue additional therapeutic intervention, e.g., additional surgery, medications, and active monitoring.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for screening patients for efficacy of an immunotherapy, the method comprising
obtaining a sample of drain fluid proximal to a tumor;
evaluating a biomarker indicative of a response to the immunotherapy; and
categorizing a patient as a candidate for the immunotherapy based on the evaluating step.

2. The method of claim 1, wherein the immunotherapy is a neoadjuvant immunotherapy.

3. The method of claim 1, wherein the immunotherapy is a checkpoint inhibitor therapy.

4. The method of claim 3, wherein the checkpoint inhibitor therapy is selected from the group consisting of PD-1 blockers, CLTA-4 blockers, and a combination of PD-1 and CLTA-4 blockers.

5. A method for evaluating the efficacy of checkpoint inhibitor therapy, the method comprising
obtaining a sample of patient lymphatic drain fluid during a surgical resection of a tumor;
performing an assay on the lymphatic drain fluid to identify a biomarker associated with an immunotherapy outcome;
obtaining a lymphatic fluid sample from the patient at a time subsequent to administration of immunotherapy; and
evaluating the biomarker to indicate an effect of the immunotherapy.

6. The method of claim 5, wherein the sample is obtained during a surgical resection.
